(19) United States
(12) Patent Application Publication
(10) Pub. No.: US 2025/0288922 A1
(43) Pub. Date: Sep. 18, 2025

(54) EXTRACTION COLUMN HAVING ALTERNATING COMPARTMENT HEIGHTS

(71) Applicant: IFP ENERGIES NOUVELLES, Rueil-Malmaison (FR)

(72) Inventors: Frederic AUGIER, Rueil-Malmaison Cedex (FR); Hélène LORCET, Rueil-Malmaison Cedex (FR)

(73) Assignee: IFP ENERGIES NOUVELLES, Rueil-Malmaison (FR)

(21) Appl. No.: 18/863,174

(22) PCT Filed: Apr. 26, 2023
(86) PCT No.: PCT/EP2023/060980

§ 371 Date: Nov. 5, 2024

(30) Foreign Application Priority Data
May 6, 2022 (FR) FR2204319

(51) Int. Cl. B01D 11/04; B01D 11/00; C07C 7/10

(52) U.S. Cl. CPC B01D 11/043; B01D 11/0492; C07C 7/10; B01D 2011/002

(57) ABSTRACT

A column for the liquid-liquid extraction of a feedstock by an extraction solvent, containing sieve trays (Pᵢ) for a dispersed phase (B) to pass through, the sieve trays being spaced apart by an inter-tray space (8), and riser/downcomer conduits (6), a riser/downcomer conduit being an opening that allows a continuous phase (A) to pass through a sieve tray, the extraction column (1) containing, in alternation, type-I sieve trays containing two peripheral riser/downcomer conduits, and type-II sieve trays containing a single central riser/downcomer conduit, wherein: the height H1 of the inter-tray spaces situated directly downstream of the type-I trays, in the direction of flow of the continuous phase, is greater than the height H2 of the inter-tray spaces positioned directly downstream of the type-II trays. Also, a liquid-liquid extraction method that makes use of the liquid-liquid extraction column.

### TECHNICAL FIELD

The field of the invention is that of liquid-liquid extraction in sieve-tray columns with riser/downcomer conduits, as are used in numerous chemical processes including processes for extracting the aromatics present in various petrochemical cuts or deriving from refinery processes. Liquid-liquid extraction columns are notably suitable for the liquid-liquid separation of hydrocarbon compounds, such as aromatic (e.g. A6-A11) compounds originating from broad hydrocarbon cuts (e.g. C6-C11 cut), such as originating from a catalytic cracking (FCC, Fluid Catalytic Cracking) unit.

### PRIOR ART

The principle of operation of liquid-liquid extraction columns is based on the differences in solubility of the compounds of a homogeneous liquid feedstock in an appropriate solvent (e.g. a polar aprotic solvent, such as sulfolane or DMSO). The addition to the feedstock of a partially miscible solvent causes the appearance of a second phase to which a portion of the compounds (e.g. the aromatic compounds), which are the most soluble constituents, is preferentially transferred.

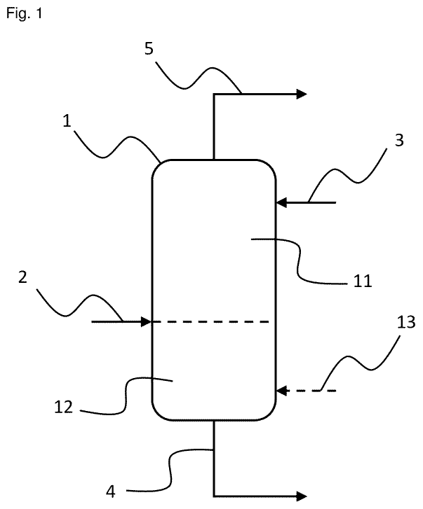

With reference to FIG. 1, a liquid-liquid extraction column 1 comprises the following elements:
a first injection point 2 for injection of a first phase (liquid feedstock that is to be separated);
a second injection point 3 for injection of a second phase (the liquid separation solvent), the first and second injection points 2 and 3 being positioned in such a way as to allow the first and second phases to circulate in a countercurrent manner;
a first withdrawal point for withdrawal of an extract 4 (in liquid phase), such as a solvent enriched in compounds extracted from the feedstock; and
a second withdrawal point for withdrawal of a raffinate 5 (in liquid phase), such as a feedstock impoverished in extracted compounds, one of the first and second withdrawal points being located at the bottom of the extraction column 1 and the other at the top of the extraction column 1. Furthermore, the first injection point 2 for injection of the feedstock that is to be separated is typically located between the second injection point 3 for injection of the liquid separation solvent and the first withdrawal point for withdrawal of the extract 4. Likewise, the second injection point 3 for injection of the liquid separation solvent is typically located between the first injection point 2 for injection of the feedstock that is to be separated and the second withdrawal point for withdrawal of the raffinate 5. In this reference example, the first phase is the light phase and the second phase is the heavy phase.

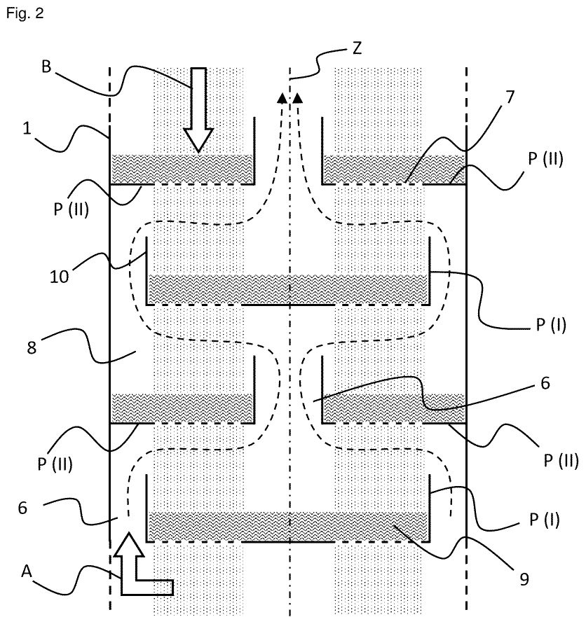

With reference to FIG. 2, a liquid-liquid extraction column 1 comprises a plurality of sieve trays P, each sieve tray P being equipped with from one to several riser/downcomer conduits 6 depending on the intended capacities, such a conduit 6 being an opening that allows the first phase A, referred to as “continuous” to pass through the sieve tray P. A sieve tray P comprises at least one zone perforated with holes 7 through which the second phase B, referred to as “dispersed”, circulates. The dispersed second phase B circulates in the form of droplets that pass through the continuous first phase A in each inter-tray space 8 defined by the space between two adjacent sieve trays P. In the example of FIG. 2, the dispersed second phase B is the heavy phase and accumulates above the sieve tray P, until it forms a coalesced layer 9 above the sieve tray. This coalesced layer 9 may typically measure from 1 cm to 8 cm in thickness. It must be appreciated that if the dispersed phase is the light phase, the dispersed phase accumulates beneath the sieve tray. The dispersed second phase B then passes through the perforated zone of the sieve tray P to feed the next sieve tray P (the tray below if the dispersed phase is the heavy phase), and so on. The continuous first phase A follows a sinuous path through the inter-tray spaces 8, passing via the conduits 6, in an upward direction in this example of FIG. 2 because the continuous phase is the light phase.

U.S. Pat. Nos. 3,632,315 and 4,588,563 describe examples of liquid-liquid extraction columns comprising sieve trays equipped with one and/or two riser/downcomer conduits.

Thus, a liquid-liquid extraction column allows two partially immiscible liquid phases A and B to be brought into contact with one another in a countercurrent manner. Depending on the choice of the dispersed phase (heavy phase or light phase), the sieve tray P is designed to collect (by means of weir plates 10) the coalesced layer 9 on the upper part of the sieve tray (when the dispersed phase is the heavy phase) or on the lower part (when the dispersed phase is the light phase). In the figures of the present description, only the scenario in which the liquid separation solvent is the dispersed phase and the heavy phase is described.

The Applicant Company has identified that the operation of a liquid-liquid extraction column can give rise to substantial variability in performance, which is notably dependent on:
the surface area of interface between the phases in the inter-tray spaces, which is dependent on the size of the droplets of the dispersed phase and on the volume fraction thereof; and
the flow of each phase in the inter-tray spaces, the riser/downcomer conduits and the perforated zones.

It is known to those skilled in the art, and perfectly described in works on liquid-liquid extraction (see Handbook of Solvent Extraction, by Lo, Baird and Hanson, Krieger Publishing Company, 1991), that plug flow of each phase is favourable to transfer performance in countercurrent extraction columns. Plug flow is a conventional concept which considers that, over a given section of the column, each phase (heavy or light) circulates at a uniform velocity and in just one direction. It is also known that the presence of any recirculation, vortex or any other movement of liquid that gives rise to a spatial velocity distribution, can lead to plug flow degradation. Flow degradations, or deviations from pure plug flow, are grouped under the generic heading of “axial mixing”. Axial dispersion may also be referred to. The effect of the axial mixing tends to homogenize the concentration gradients present in one or other phase along the column, and tends to minimize the efficiency of the liquid-liquid extraction.

In particular, recirculations of continuous phase in the riser/downcomer conduits are very detrimental. As regards the flow of the dispersed phase, it is essential for the flow to remain essentially downward (or upward) through the perforated zones. Any entrainment of dispersed phase by the continuous phase in the riser/downcomer conduits would be detrimental because this would tend to reduce the difference in concentrations of the continuous phase in the successive inter-tray spaces. For this reason, a person skilled in the art uses riser/downcomer conduits that are large enough to minimize the entrainment of the dispersed phase in these conduits. To avoid using up too much surface area, it is also preferable for the cross section of the riser/downcomer conduits not to be too excessive either; for a given diameter, oversizing the conduit surface area may lead to:
a reduction in the capacity of the column (and therefore an increase in the cost thereof, for the same performance),
a reduction in the extraction efficiency: by reducing the remaining surface area available for the dispersion of the heavy phase, the quality of the contact between the phases is degraded,
the generation of recirculations of continuous phase in the riser/downcomer conduits themselves, a reduction in the plug-flow nature of the flow of the continuous phase and thus a reduction in the extraction efficiency.

The cross section of the riser/downcomer conduits my for example be chosen so that the velocity of the continuous phase in these conduits does not exceed the terminal velocity at which droplets of a diameter smaller than the mean droplet diameter fall. For example, a fine-droplets diameter of 30 μm to 150 μm may be considered for calculating the maximum velocity of the light phase in the riser/downcomer conduits.

Another aspect needing careful consideration is the impact of the flow of the continuous phase in each inter-tray space. The flow of the continuous phase is governed by the circulating continuous-phase flow rate and by the geometry of the inter-tray spaces, notably including the height of the inter-tray space.

If the continuous phase circulates through the column too quickly, there are various detrimental behaviours that may occur, such as:
disturbances in the flow of the dispersed phase, which may be entrained by the continuous phase towards the riser/downcomer conduit;
deformation of the layer that has coalesced on the tray that is downstream in the direction of flow of the dispersed phase, this deformation also tending to cause dispersed phase to accumulate under the inter-tray space outlet conduit.

These two behaviours may encourage the presence of dispersed phase in the riser/downcomer conduits and potentially cause it to be entrained by the continuous phase towards the inter-tray space downstream in the direction of flow of the continuous phase. Specifically, the presence of dispersed phase in the riser/downcomer conduit of a tray P is detrimental to the extraction performance because it may give rise to a back-mixing of dispersed phase and substantially reduce the height of coalesced layer on tray P+1 (the tray downstream in the direction of flow of the dispersed phase). Furthermore, the reduction in the height of the coalesced layer may cause a partial drying-out of the sieve trays, leading to nonuniformities in the distribution of the dispersed phase in the inter-tray space downstream in the direction of flow of the dispersed phase, or even cause some continuous phase to pass through the trays, a phenomenon which increases axial dispersion and reduces column efficiency.

In order to keep the flow of the dispersed phase at a velocity level that is not problematical, there are a number of solutions that may be implemented, such as:
increasing the height of the inter-tray space;
increasing the number of riser/downcomer conduits on each tray.

Increasing the height of the compartments makes it possible effectively to reduce the entrainment of heavy phase towards the outlet riser/downcomer conduit, but naturally increases the height of the extraction columns, something which has an associated cost and cannot be pushed excessively high.

Increasing the number of riser/downcomer conduits is highly effective, because that allows local lateral splitting of the circulating stream of dispersed phase. Trays having 1 riser/downcomer conduit are generally reserved for columns of small size (diameter <2 m). Trays having 2 riser/downcomer conduits are very conventional and are effective at reducing the velocity of the light phase. Trays having larger numbers of riser/downcomer conduits are also possible.

Increasing the number of riser/downcomer conduits increases the non-perforated cross section of the column, and therefore leads to a loss in volume useable for bringing the phases into contact. For this reason, it is not economically sensible to increase the number of riser/downcomer conduits beyond the minimum required to prevent entrainment of the heavy phase into these conduits.

With reference to FIG. 2, a conventional implementation of liquid-liquid extraction columns comprising trays with 2 riser/downcomer conduits is to use an alternation of 2 distinct trays so that the flow of the continuous phase circulates along a sinuous path in the column passing through zones in which droplets of the dispersed phase are present. This makes it possible to increase the transfer of matter between the phases. A first type of tray (type I) uses 2 peripheral or lateral riser/downcomer conduits (orthogonal to the central/vertical axis Z of the column), substantially bonded to the shell of the column and arranged in diametrically opposite positions. The second type of tray (type II) uses just 1 central riser/downcomer conduit, in the form of a strip (perpendicular to the central/vertical axis Z of the column) covering substantially the entire diameter of the column. A liquid-liquid extraction column as depicted in FIG. 2 is a column said to be a 2-pass column, i.e. a column in which the continuous phase circulates following a sinuous path along 2 different routes as indicated by the dotted-line arrows in FIG. 2.

In the present description, when the dispersed phase is the heavy phase, the inter-tray space of a type-I tray is the inter-tray space situated directly above the type-I tray; the inter-tray space of a type-II tray is the inter-tray space situated directly above the type-II tray. Thus, the inter-tray space corresponds to the space located on the side of the weir plate 10 and of the coalesced layer 9 of the tray Pᵢ, i.e. the inter-tray space of a tray Pᵢ corresponds to the space located downstream of the tray Pᵢ in the direction of flow of the continuous phase.

The prior art consists in using alternating trays of types I and II, with a common inter-tray space height and identical riser/downcomer conduit cross sections, the central riser/downcomer conduit of the type-II trays then having a cross section equal to the sum of the 2 riser/downcomer conduits of the type-I trays.

It is an object of the present invention to overcome the above-mentioned performance deficiencies and to increase the performance of liquid-liquid extraction columns.

### SUMMARY OF THE INVENTION

In the context described above, a first object of the present description is to provide a liquid-liquid extraction column which makes it possible:
to maintain a range of between 5% and 20% of the mean volume fraction of dispersed phase (e.g., solvent/heavy phase) in a compartment (i.e. zone comprising a sieve tray and an adjacent inter-tray space);
the non-entrainment of the droplets of dispersed phase into the riser/downcomer conduits by the continuous phase (e.g. feedstock/light phase), in order to limit the axial mixing of the dispersed phase;
a height of coalesced layer of the dispersed phase on each tray which is sufficient (e.g. at least 2 cm, such as from 2 cm to 4 cm) to prevent the passage of the continuous phase through the sieve tray (and to force the exclusive passage of the continuous phase into the riser/downcomer conduits);
a suitable continuous phase transverse velocity, which does not perturb the flow of the dispersed phase.

Surprisingly, the Applicant Company has identified that specific characteristics of sieve trays, such as the height of the inter-tray spaces and optionally the cross section of the riser/downcomer conduits, make it possible to control the hydrodynamics along the entire column, while limiting the axial mixing. This technical solution makes it possible to maintain a satisfactory material transfer efficiency on each tray.

According to a first aspect, the abovementioned objects, and also other advantages, are obtained by a liquid-liquid extraction column, comprising the following elements:
a first injection point for injection of a first phase;
a second injection point for injection of a second phase, the first and second injection points being positioned on the extraction column in such a way as to allow the first and second phases to circulate in the extraction column in a countercurrent manner, one of the first and second phases being a continuous phase and the other being a dispersed phase;
a first withdrawal point for withdrawal of an extract and a second withdrawal point for withdrawal of a raffinate, one being located at the bottom of the extraction column and the other being located at the top of the extraction column; and
a plurality of sieve trays located from the top of the extraction column to the bottom of the extraction column, the sieve trays being spaced apart by an inter-tray space and comprising weir plates designed to hold a layer of the dispersed phase that has coalesced above or below the sieve trays;
a plurality of riser/downcomer conduits, such a conduit being an opening adjacent to a weir plate and allowing the continuous phase to pass through the sieve tray,
   the extraction column comprising in alternation:
sieve trays said to be of type I, comprising two peripheral riser/downcomer conduits; and
sieve trays said to be of type II, comprising a single central riser/downcomer conduit,
   in which extraction column:
the height H1 of the inter-tray spaces situated directly downstream of the type-I trays, in the direction of flow of the continuous phase, is greater than the height H2 of the inter-tray spaces positioned directly downstream of the type-II trays.

According to one or more embodiments, the ratio H1/H2 of the height H1 to the height H2 is comprised between 1.1 and 2.

According to one or more embodiments, the ratio H1/H2 of the height H1 to the height H2 is comprised between 1.1 and 1.5.

According to one or more embodiments, the height H1 of the inter-tray spaces of the type-I sieve trays is comprised between 0.22 m and 1.50 m, preferably between 0.27 m and 0.90 m, very preferably between 0.33 m and 0.82 m.

According to one or more embodiments, the height H2 of the inter-tray spaces of the type-II sieve trays is comprised between 0.20 m and 1.00 m, preferably between 0.25 m and 0.60 m, very preferably between 0.30 m and 0.55 m.

According to one or more embodiments, the liquid-liquid extraction column comprises:
an extraction sector, extending substantially from the first injection point for injection of the first phase as far as substantially the second injection point for injection of the second phase, and
a backwash sector, adjacent to the extraction sector and extending from the first injection point for injection of the first phase as far as substantially a third injection point for injection of a backwash liquid.

According to one or more embodiments, the cross section S2 of the central riser/downcomer conduits is greater than the cross section S1 of the peripheral riser/downcomer conduits, the cross section S1 corresponding to the sum of the cross sections of the two peripheral riser/downcomer conduits.

According to one or more embodiments, the ratio S2/S1 of the cross section S2 to the cross section S1 is comprised between 1.1 and 2.

According to one or more embodiments, the ratio S2/S1 of the cross section S2 to the cross section S1 is comprised between 1.1 and 1.5.

According to a second aspect, the abovementioned objects, and also other advantages, are obtained by a liquid-liquid extraction method comprising the following steps:
injecting a first phase and a second phase into a liquid-liquid extraction column according to the first aspect; and
withdrawing an extract and a raffinate from the liquid-liquid extraction column.

According to one or more embodiments, the extraction column is operated at a pressure comprised between 0.05 MPa and 3 MPa, preferably between 0.1 MPa and 2 MPa, preferably between 0.2 MPa and 1.5 MPa, and preferably between 0.3 MPa and 1 MPa; and at a temperature comprised between 10° C. and 150° C., preferably between 15° C. and 130° C., preferably between 30° C. and 120° C., and preferably between 40° C. and 110° C.

According to one or more embodiments, the first phase contains a mixture of aromatic and nonaromatic compounds.

According to one or more embodiments, the second phase contains a compound selected from ethylene glycol, diethylene glycol, triethylene glycol, hexamethylphosphoramide, propylene carbonate, ethylene carbonate, sulfolane, 3-methylsulfolane, N-methylacetamide, N,N-dimethylacetamide, butyrolactone, 1-methylpyrrolidone, dimethyl sulfoxide, caprolactam, N-methylformamide, pyrrolidin-2-one, furfural, 1,1,3,3-tetramethylurea and a mixture of these.

According to one or more embodiments, the second phase comprises sulfolane and water.

Embodiments of the liquid-liquid extraction column and method according to the aforementioned aspects, and also other features and advantages, will become apparent from reading the following description, which is given purely by way of non-limiting illustration, and with reference to the following drawings.

### LIST OF THE FIGURES

The FIG. 1 schematically shows a cross-sectional view of a liquid-liquid extraction column.

The FIG. 2 schematically shows a cross-sectional view of the flow of the dispersed phase and of the continuous phase in the reference liquid-liquid extraction column.

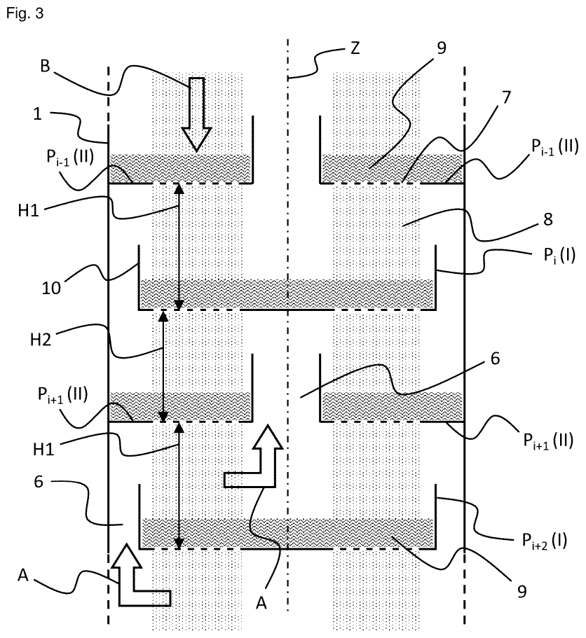

The FIG. 3 schematically shows a cross-sectional view of the flow of the dispersed phase and of the continuous phase in a liquid-liquid extraction column according to the present invention.

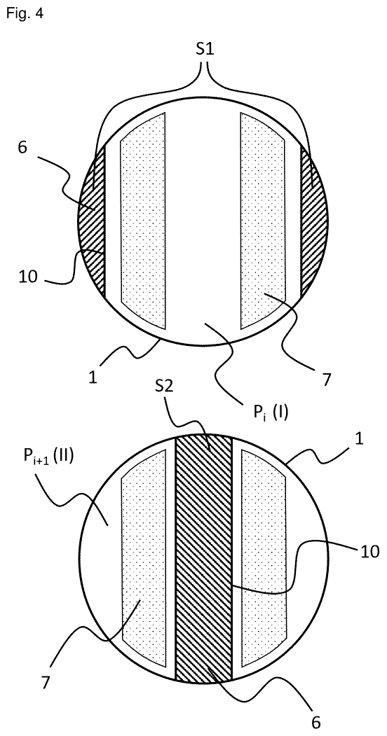

The FIG. 4 schematically shows a top view of a type-I sieve tray and a type-II sieve tray according to the present invention.

### DESCRIPTION OF THE EMBODIMENTS

Embodiments of the invention will now be described in detail. In the following detailed description, many specific details are presented in order to provide a deeper understanding of the invention. However, it will be apparent to a person skilled in the art that the invention can be implemented without these specific details. In other cases, well-known characteristics have not been described in detail in order to avoid unnecessarily complicating the description.

In the present description, the term “to comprise” is synonymous with (means the same thing as) “to include” and “to contain”, and is inclusive or open-ended and does not exclude other elements that are not stated. It is understood that the term “to comprise” includes the exclusive and closed term “to consist”. In addition, in the present description, the term “substantially” corresponds to an approximation of ±10%, preferably of ±5%, very preferably of ±2%, of a reference value, such as a distance, a velocity, a flow rate, a content of compounds, a temperature, a pressure, etc.

### Liquid-Liquid Extraction Column

A liquid-liquid extraction column according to the present invention comprises all the elements defined in FIG. 1. In addition, in order to increase the yield and the purity, two distinct operating zones can be defined in a liquid-liquid extraction column according to the present invention opposite the first injection point 2 for injection of the liquid to be separated:
an extraction sector 11, extending substantially from the first injection point 2 for injection of the first phase up to substantially the second injection point 3 for injection of the second phase, makes it possible notably to extract compounds (e.g. aromatics) from the liquid to be separated by making contact with the liquid separation solvent (the “yield” zone) in a countercurrent manner, and
an optional backwash sector 12, adjacent to the extraction sector 11 and extending from the first injection point 2 for injection of the first phase up to substantially a third injection point 13 for injection of a backwash liquid (optional), such as a recycled extract, makes it possible notably to back-extract undesired compounds (e.g. heavy nonaromatic compounds) contained in the extract 4 by way of the backwash liquid in order to guarantee a high level of purity. According to one or more embodiments, the third injection point 13 for injection of a backwash liquid is arranged between the first injection point 2 and the first withdrawal point for withdrawal of the extract 4.

Specifically with reference to FIG. 1, the separation liquid exits from the liquid-liquid extraction column 1 while entraining compounds of interest that are to be separated (e.g. aromatic compounds) to form the extract 4. The extract may also contain undesired compounds (e.g. light nonaromatic compounds, such as C6-C7 compounds) which can be separated downstream (e.g. by distillation and/or stripping). Advantageously, the extract 4 contains substantially no (or very few) undesired compounds which are difficult to separate (e.g. heavier nonaromatic compounds, such as C8+ compounds) and are separated from the extract in the backwash sector 12. With reference to FIG. 1, the separation liquid is heavier than the liquid to be separated and is injected at the top of the liquid-liquid extraction column 1, while the optional backwash liquid is injected at the bottom of the liquid-liquid extraction column 1. It is understood that the present invention also relates to liquid-liquid extraction columns in which the separation liquid is lighter than the liquid to be separated, the injection point 3 for injection of the separation liquid then being at the bottom of the liquid-liquid extraction column 1 and the injection point 13 for injection of the backwash liquid being at the top of the liquid-liquid extraction column 1.

With reference to FIGS. 3 and 4, a liquid-liquid extraction column 1 according to the present invention comprises n sieve trays Pᵢ, i being comprised between 1 and n. Each sieve tray Pᵢ is located so that the dispersed phase (i.e. the separation liquid heavier than the liquid to be separated) flows through the holes 7 in the sieve tray Pᵢ, the droplets of the dispersed phase B recoalescing on the next sieve tray Pᵢ₊₁ to form a liquid volume preventing the passage of the continuous phase A (i.e. the liquid to be separated lighter than the separation liquid) through the holes in the sieve tray Pᵢ₊₁. The liquid to be separated circulates in a countercurrent manner in relation to the separation liquid, i.e. from the bottom to the top through the central and peripheral riser/downcomer conduits 6, and transversely in an inter-tray space 8. With reference to FIG. 3, the heavy phase is the dispersed phase B and the light phase is the continuous phase A. It is understood that a liquid-liquid extraction column 1 can comprise sieve trays designed for the dispersed phase to be the light phase and the continuous phase to be the heavy phase.

According to one or more embodiments, the number n of sieve trays Pᵢ is comprised between 10 and 200, preferably between 50 and 150.

According to the invention, the liquid-liquid extraction column 1 alternatively comprises type-II sieve trays Pᵢ, i.e. comprising just one central riser/downcomer conduit, and type-I sieve trays Pᵢ, i.e. comprising two peripheral riser-downcomer conduits.

Specifically, the type-II sieve trays have a zone perforated with holes 7 that is divided into 2 portions, on either side of the central riser/downcomer conduit 6, as shown on the tray Pᵢ in FIG. 4. Moreover, the type-I sieve trays have a zone perforated with holes 7 that can be unique or divided into 2 portions, as presented on the plate Pᵢ₊₁ in FIG. 4, in order to remain similar to the portions of the zone perforated with holes 7 of the type-II sieve trays.

As a result, it is understood that the present invention relates to 2-pass liquid-liquid extraction columns. Specifically, with reference to FIGS. 3 and 4, the liquid-liquid extraction column 1 comprises trays Pᵢ and Pᵢ₊₂ with 2 peripheral riser/downcomer conduits 6 (type-I trays) arranged in alternation with the trays Pᵢ₋₁ and Pᵢ₊₁ with 1 central riser/downcomer conduit 6 (type-II trays). Preferably, the peripheral riser/downcomer conduits 6 are substantially adjacent to the shell of the column, or even substantially bonded to the said shell. Preferably, the peripheral riser/downcomer conduits 6 are substantially arranged in diametrically opposite positions. Preferably, the central riser/downcomer conduits 6 are arranged in the form of a strip covering substantially the entire diameter of the column.

The Applicant Company has identified, notably in the course of a numerical study of the flow in the alternating inter-tray spaces of the type-I and type-II sieve trays Pᵢ, that an entrainment of dispersed phase into the riser/downcomer conduits was possible and could minimize the extraction performance. It was observed that this entrainment occurred earliest in the riser/downcomer conduits of the type-II trays (central riser/downcomer conduits).

According to the invention, the sieve trays are modified in a differentiated way depending on their type. Whereas the prior art would propose to increase the height of the inter-tray spaces and the cross section (surface area) of riser/downcomer conduits of all the trays, the Applicant Company has identified that the entrainment of dispersed phase in the continuous phase could be reduced by:
increasing the height H1 of the inter-tray spaces 8 of the type-I sieve trays Pᵢ to a level greater than the height H2 of the inter-tray spaces 8 of the type-II sieve trays Pᵢ, as shown in FIG. 3; and
optionally increasing the cross section S2 of the central riser/downcomer conduits 6 of the type-II sieve trays Pᵢ to a level greater than the cross section S1 of the peripheral riser/downcomer conduits 6 of the type-I sieve trays Pᵢ, as shown in FIG. 4.

It is understood in the present description that the height of an inter-tray space 8 of a sieve tray Pᵢ corresponds to the distance between the said sieve tray Pᵢ and the sieve tray arranged directly downstream in the direction of flow of the continuous phase, i.e. the sieve tray Pᵢ₋₁ in the example of FIG. 3.

It is understood in the present description that the cross section S1 of the peripheral riser/downcomer conduits 6 of a sieve tray corresponds to the sum of the cross sections of the two peripheral riser/downcomer conduits of the said sieve tray.

Advantageously, by alternating type-I and type-II sieve trays in the extraction column 1, the present invention makes it possible to avoid the entrainment of dispersed phase in the riser/downcomer conduits of the type-II plates (central riser/downcomer conduits) whilst still minimizing the drawbacks of the modifications applied. As a result, the increase in the column height is reduced by 2 in relation to the prior art, in which the heights of all the inter-tray spaces are increased.

Similarly, the increase in the cross section of the riser/downcomer conduits of the type-II sieve trays can be accompanied by a reduction in the perforated surface area and in the transfer quality in the inter-tray space of the type-I sieve trays, but this reduction does not concern the inter-tray spaces of the type-II sieve trays. Therefore, the impact of the increase in the cross section of the riser/downcomer conduits of the type-II sieve trays degrades the transfer performance less than does a change in cross section over all of the riser/downcomer conduits, according to the prior art.

According to one or more embodiments, the ratio H1/H2 of the height H1 to the height H2 is comprised between 1.1 and 2, preferably between 1.1 and 1.5. According to one or more embodiments, the ratio H1/H2 of the height H1 to the height H2 is comprised between 1.2 and 1.9, preferably between 1.3 and 1.7.

According to one or more embodiments, the height H1 of the inter-tray spaces 8 of the type-I sieve trays is comprised between 0.22 m and 1.50 m, preferably between 0.27 m and 0.90 m, very preferably between 0.33 m and 0.83 m.

According to one or more embodiments, the height H2 of the inter-tray spaces 8 of the type-II sieve trays is comprised between 0.20 m and 1.00 m, preferably between 0.25 m and 0.60 m, very preferably between 0.30 m and 0.55 m.

According to one or more embodiments, the ratio S2/S1 of the cross section S2 to the cross section S1 is comprised between 1.1 and 2, preferably between 1.1 and 1.5. According to one or more embodiments, the ratio S2/S1 of the cross section S2 to the cross section S1 is comprised between 1.2 and 1.4.

According to one or more embodiments, the cross section of the riser/downcomer conduits 6 is predetermined such that the velocity of the continuous phase passing through the said riser/downcomer conduits is comprised between 0.005 m/s and 0.050 m/s. According to one or more embodiments, the flow rate of the continuous phase is comprised between 70 m³/hr and 400 m³/hr, preferably between 80 m³/hr and 350 m³/hr.

According to one or more embodiments, the cross sections S1 and S2 of the riser/downcomer conduits 6 are comprised between 0.3 m² and 15 m², preferably between 2 m² and 10 m². According to one or more embodiments, the cross section S1 of the central riser/downcomer conduit 6 is comprised between 0.33 m² and 15 m², preferably between 2.2 m² and 10 m². According to one or more embodiments, the cross section S2 of the peripheral riser/downcomer conduits 6 is comprised between 0.3 m² and 13.5 m², preferably between 2 m² and 9 m².

The diameter of the extraction column 1 typically depends on the predetermined value of the flow rate of the dispersed phase passing through it. According to one or more embodiments, the flow rate of the dispersed phase is comprised between 140 m³/hr and 2300 m³/hr, preferably between 200 m³/hr and 2000 m³/hr.

The height of the extraction column 1 typically depends on its diameter. According to one or more embodiments, the height of the extraction column is comprised between 15 m and 90 m.

According to one or more embodiments, the sieve trays Pᵢ have holes 7 with a diameter comprised between 2 mm and 10 mm. According to one or more embodiments, the diameter of the holes is predetermined such that the velocity at the hole is comprised between 0.05 m/s and 0.60 m/s.

### Liquid-Liquid Extraction Method

According to one or more embodiments, the extraction column 1 is operated at a pressure comprised between 0.05 MPa and 3 MPa (0.5 and 30 bara), preferably between 0.1 MPa and 2 MPa (1 and 20 bara), preferably between 0.2 MPa and 1.5 MPa (2 and 15 bara), preferably between 0.3 MPa and 1 MPa (3 and 10 bara). According to one or more embodiments, the extractor is operated at a temperature comprised between 10° C. and 150° C., preferably between 15° C. and 130° C., preferably between 30° C. and 120° C., preferably between 40° C. and 110° C.

### Liquid Feedstock to be Separated

The liquid-liquid extraction method according to the invention makes it possible to treat a liquid feedstock to be separated comprising a mixture of aromatic and nonaromatic compounds. Preferably, the feedstock is hydrotreated and/or hydrogenated. According to one or more embodiments, the feedstock is an optionally hydrotreated and/or hydrogenated petrol feedstock.

According to one or more embodiments, the feedstock is a C5+ cut, i.e. containing compounds having 5 and more carbon atoms. According to one or more embodiments, the feedstock is a C5-C10 or C5-C11 cut, i.e. a cut containing compounds comprising from 5 to 10 or from 5 to 11 carbon atoms. According to one or more embodiments, the feedstock is a C6-C10 or C6-C11 cut, i.e. a cut containing compounds comprising from 6 to 10 or from 6 to 11 carbon atoms.

According to one or more embodiments, the feedstock comprises at least 20% by weight, preferably at least 30% by weight, very preferably at least 40% by weight (e.g. at least 50% by weight), of aromatic compounds with 6 to 11 carbon atoms, with respect to the total weight of the feedstock.

According to one or more embodiments, the feedstock comprises at least 20% by weight, preferably at least 30% by weight, very preferably at least 40% by weight (e.g. at least 50% by weight), of monoaromatic compounds with 6 to 11 carbon atoms, with respect to the total weight of the feedstock.

According to one or more embodiments, the aromatic compounds of the feedstock are monoaromatic compounds at least at 95% by weight, preferably at least at 98% by weight, very preferably at least at 99% by weight.

According to one embodiment of the invention, the feedstock comprises less than 50 ppm by weight of sulfur, preferably less than 10 ppm by weight of sulfur, and very preferably less than 1 ppm by weight of sulfur.

According to one embodiment of the invention, the feedstock comprises less than 100 ppm by weight of nitrogen, preferably less than 10 ppm by weight of nitrogen, and very preferably less than 1 ppm by weight of nitrogen.

According to one embodiment of the invention, the feedstock comprises less than 0.1% by weight of diolefins, preferably less than 0.05% by weight of diolefins, and very preferably less than 0.01% by weight of diolefins.

According to one embodiment of the invention, the feedstock comprises less than 0.1% by weight of olefins, preferably less than 0.05% by weight of olefins, and very preferably less than 0.01% by weight of olefins.

According to one embodiment of the invention, the feedstock exhibits a content of less than or equal to 5000 ppm by weight, preferably less than or equal to 4500 ppm by weight, and very preferably less than or equal to 3000 ppm by weight, of compounds having a boiling point greater than 217° C., such as naphthalene.

According to one embodiment of the invention, the feedstock is devoid of the following compounds: H₂, H₂S, light gas such as ethane, propane and butane. According to one embodiment of the invention, the removal of these compounds from the feedstock is carried out in a fractionation column.

According to one embodiment, the said feedstock is at least in part a petrol cut resulting from a fluidized bed catalytic cracking unit (FCC (for Fluid Catalytic Cracking) unit), the petrol cut preferably having been selectively hydrogenated in order to convert diolefins into olefins, then fractionated in order to obtain a C5-C10, C5-C11, C6-C10 or C6-C11 cut, and then hydrogenated in order to saturate the olefinic compounds. According to one embodiment of the invention, the feedstock results from the hydrogenation of a pyrolysis gasoline (PyGas) in the form of a mixture with a petrol cut resulting from an FCC unit.

### Liquid Separation Solvent

According to one or more embodiments, the solvent contains a compound selected from ethylene glycol, diethylene glycol, triethylene glycol, hexamethylphosphoramide, propylene carbonate, ethylene carbonate, sulfolane, 3-methylsulfolane, N-methylacetamide, N,N-dimethylacetamide, butyrolactone, 1-methylpyrrolidone, dimethyl sulfoxide, caprolactam, N-methylformamide, pyrrolidin-2-one, furfural, 1,1,3,3-tetramethylurea and a mixture of these.

According to one or more embodiments, the solvent comprises or consists of sulfolane. According to one or more embodiments, the solvent is made up of at least 80% by weight (e.g. of at least 90% by weight), preferably of at least 95% by weight (e.g. of at least 99% by weight), of sulfolane, with respect to the total weight of the solvent.

According to one or more embodiments, the solvent additionally comprises an anti-solvent, such as water. According to one or more embodiments, the anti-solvent comprises or consists of water. According to one or more embodiments, the solvent comprises between 0.01% by weight and 5% by weight, preferably between 0.1% by weight and 3% by weight (e.g. between 0.5% by weight and 2% by weight), of anti-solvent, such as water, with respect to the total weight of the solvent. According to one or more embodiments, the solvent comprises or consists of sulfolane and water.

### EXAMPLES

The examples of a liquid-liquid extraction column that are described below have all the following features: The liquid-liquid extraction column has a diameter of 4.8 m. Each tray comprises 17 192 holes. The feedstock (continuous and light phase) is injected at the lower tray with a flow rate of 1.02E+05 kg/hr. The feedstock comprises 40% by weight of isooctane, 30% by weight of benzene and 30% by weight of para-xylene (density of 724 kg/m³). The liquid separation solvent (dispersed and heavy phase) is injected at the upper tray with a flow rate of 8.25E+05 kg/hr. The liquid separation solvent comprises 99.5% by weight of sulfolane and 0.5% by weight of water (density of 1136 kg/m³).

### Example 1 (Reference): Constant Height of the Inter-Tray Spaces and Constant Riser/Downcomer Conduit Cross Sections

The liquid-liquid extraction column has a height of 36.0 m and is composed of a succession of 120 sieve trays.

No adjustment is made:
heights of the inter-tray spaces: H1=H2=0.30 m,
cross sections of the riser/downcomer conduits: S1=S2=1.70 m².

In the reference example 1, disturbances to the flow of the dispersed phase are observed, some of the dispersed phase is entrained with some of the continuous phase towards the riser/downcomer conduit, and deformation of the coalesced layer is observed on the tray downstream in the direction of flow of the dispersed phase. To quantitatively express the performance, the Height Equivalent to a Theoretical Plate (HETP), a conventional concept in separation (distillation, extraction), is calculated. The lower the HETP is, the more effective the extractor is.

For the reference example 1, the HETP is 4.76 m.

### Example 2 (Reference): Increase in the Height of the Inter-Tray Spaces

In relation to example 1, the heights H1 and H2 of the inter-tray spaces are increased:
heights of the inter-tray spaces: H1=H2=0.45 m,
cross sections of the riser/downcomer conduits: S1=S2=1.70 m².

For the reference example 2, the HETP is 5.83 m.

The liquid-liquid extraction column has a height of 44.0 m and is composed of a succession of 98 sieve trays.

### Example 3 (Reference): Increase in the Cross Sections of the Riser/Downcomer Conduits

In relation to example 1, the cross sections S1 and S2 of the riser/downcomer conduits are increased:
heights of the inter-tray spaces: H1=H2=0.30 m,
cross sections of the riser/downcomer conduits: S1=S2=2.10 m².

For the reference example 3, the HETP is 4.81 m.

The liquid-liquid extraction column has a height of 36.3 m and is composed of a succession of 121 sieve trays.

### Example 4 (Invention): Alternating Heights of the Inter-Tray Spaces

In relation to example 1, only the heights H1 are increased:
heights of the inter-tray spaces: H1=0.45; H2=0.30 m,
cross sections of the riser/downcomer conduits: S1=S2=1.70 m².

For example 4 according to the invention, the HETP is 4.26 m.

The liquid-liquid extraction column has a height of 32.2 m and is composed of a succession of 86 sieve trays.

### Example 5 (Invention): Alternating Heights of the Inter-Tray Spaces and Alternating Cross Sections of the Riser/Downcomer Conduits

In relation to example 1, only the heights H1 and cross sections S2 are increased:
heights of the inter-tray spaces: H1=0.45 m; H2=0.30 m,
cross sections of the riser/downcomer conduits: S1=1.70 m²; S2=2.10 m².

For example 5 according to the invention, the HETP is 4.08 m.

The liquid-liquid extraction column has a height of 30.8 m and is composed of a succession of 82 sieve trays.

Table 1 below summarizes the levels of performance of reference examples 1, 2 and 3 and examples 4 and 5 according to the invention.

TABLE 1

| Example | 1 (ref.) | 2 (ref.) | 3 (ref.) | 4 (inv.) | 5 (inv.) |
|---|---|---|---|---|---|
| H1 (m) | 0.30 | 0.45 | 0.30 | 0.45 | 0.45 |
| H2 (m) | 0.30 | 0.45 | 0.30 | 0.30 | 0.30 |
| S1 (m²) | 1.70 | 1.70 | 2.10 | 1.70 | 1.70 |
| S2 (m²) | 1.70 | 1.70 | 2.10 | 1.70 | 2.10 |
| HETP (m) | 4.76 | 5.83 | 4.81 | 4.26 | 4.08 |
| Column height (m) | 36.0 | 44.0 | 36.3 | 32.3 | 30.8 |
| Number of trays | 120 | 98 | 121 | 86 | 82 |

Advantageously, the HETP, the column height, and the number of trays of examples 4 and 5 according to the invention are reduced in relation to those of reference examples 1, 2 and 3.

1. Liquid-liquid extraction column comprising the following elements:
a first injection point (2) for injection of a first phase;
a second injection point (3) for injection of a second phase, the first and second injection points (2, 3) being positioned on the extraction column (1) in such a way as to allow the first and second phases to circulate in the extraction column (1) in a countercurrent manner, one of the first and second phases being a continuous phase (A) and the other being a dispersed phase (B);
a first withdrawal point for withdrawal of an extract (4) and a second withdrawal point for withdrawal of a raffinate (5), one being located at the bottom of the extraction column (1) and the other being located at the top of the extraction column (1); and
a plurality of sieve trays (Pᵢ) located from the top of the extraction column (1) to the bottom of the extraction column (1), the sieve trays (Pᵢ) being spaced apart by an inter-tray space (8) and comprising weir plates (10) designed to hold a layer (9) of the dispersed phase (B) that has coalesced above or below the sieve trays (Pᵢ);
a plurality of riser/downcomer conduits (6), a riser/downcomer conduit (6) being an opening adjacent to a weir plate (10) and allowing the continuous phase (A) to pass through the sieve tray (Pᵢ),
the extraction column (1) comprising in alternation:
sieve trays (Pᵢ) said to be of type I, comprising two peripheral riser/downcomer conduits (6); and
sieve trays (Pᵢ) said to be of type II, comprising a single central riser/downcomer conduit (6), in which extraction column (1):
the height H1 of the inter-tray spaces (8) situated directly downstream of the type-I trays, in the direction of flow of the continuous phase, is greater than the height H2 of the inter-tray spaces (8) positioned directly downstream of the type-II trays.

2. Liquid-liquid extraction column according to claim 1, wherein the ratio H1/H2 of the height H1 to the height H2 is comprised between 1.1 and 2.

3. Liquid-liquid extraction column according to claim 1, wherein the ratio H1/H2 of the height H1 to the height H2 is comprised between 1.1 and 1.5.

4. Liquid-liquid extraction column according to claim 1, wherein the height H1 of the inter-tray spaces (8) of the type-I sieve trays is comprised between 0.22 m and 1.50 m, preferably between 0.27 m and 0.90 m, very preferably between 0.33 m and 0.83 m.

5. Liquid-liquid extraction column according to claim 1, wherein the height H2 of the inter-tray spaces (8) of the type-II sieve trays is comprised between 0.20 m and 1.00 m, preferably between 0.25 m and 0.60 m, very preferably between 0.30 m and 0.55 m.

6. Liquid-liquid extraction column according to claim 1, comprising:
an extraction sector (11), extending substantially from the first injection point (2) for injection of the first phase as far as substantially the second injection point (3) for injection of the second phase, and
a backwash sector (12), adjacent to the extraction sector (11) and extending from the first injection point (2) for injection of the first phase as far as substantially a third injection point (13) for injection of a backwash liquid.

7. Liquid-liquid extraction method comprising the following steps:
injecting a first phase and a second phase into the liquid-liquid extraction column according to claim 1; and
withdrawing an extract (4) and a raffinate (5) from the liquid-liquid extraction column.

8. Liquid-liquid extraction method according to claim 7, wherein the extraction column (1) is operated at a pressure comprised between 0.05 MPa and 3 MPa, preferably between 0.1 MPa and 2 MPa, preferably between 0.2 MPa and 1.5 MPa, and preferably between 0.3 MPa and 1 MPa; and at a temperature comprised between 10° C. and 150° C., preferably between 15° C. and 130° C., preferably between 30° C. and 120° C., and preferably between 40° C. and 110° C.

9. Liquid-liquid extraction method according to claim 7, wherein the first phase comprises a mixture of aromatic and nonaromatic compounds.

10. Liquid-liquid extraction method according to claim 7, wherein the second phase contains a compound selected from ethylene glycol, diethylene glycol, triethylene glycol, hexamethylphosphoramide, propylene carbonate, ethylene carbonate, sulfolane, 3-methylsulfolane, N-methylacetamide, N,N-dimethylacetamide, butyrolactone, 1-methylpyrrolidone, dimethyl sulfoxide, caprolactam, N-methylformamide, pyrrolidin-2-one, furfural, 1,1,3,3-tetramethylurea and a mixture of these.
